(19) United States
(12) Patent Application Publication
(10) Pub. No.: US 2006/0223073 A1
(43) Pub. Date: Oct. 5, 2006

(54) Methods of using a DNase I-like enzyme

(76) Inventors: Barry E. Boyes, Wilmington, DE (US); Rhonda R. Taylor, Smyma, DE (US); John R. Link, Wilmington, DE (US)

Correspondence Address: AGILENT TECHNOLOGIES, INC.;Legal Department, DL429, Intellectual Property Administration, P. O. Box 7599, Loveland, CO, 80537-0599, US

(21) Appl. No.: 11/097,868

(22) Filed: Mar. 31, 2005

(51) Int. Cl. C12Q 1/68; C12P 19/34; C12N 9/22

(52) U.S. Cl. 435/6; 435/91.2; 435/199

(57) ABSTRACT

Methods for expanding conditions of use of a DNase I-like enzyme are disclosed as are compositions and kits comprising a DNAse I-like enzyme. Compositions comprising storage-stable forms of a DNase I-like enzyme and methods for producing such compositions are also disclosed.

### BACKGROUND

Deoxyribonuclease I (DNase I) is an enzyme that is used in both research and clinical setting, e.g., in the treatment of cystic fibrosis (Ramsey, New Engl. J. Med. 1996; 335:179-188). The enzyme is a DNA endonuclease which catalyzes the hydrolysis of double-stranded DNA (dsDNA) by a double-strand or single-strand nick, leading to the depolymerization of DNA. The activity of the enzyme is maximal over a pH range of 6-9, dependant on the presence of divalaent cations, such as Ca⁺², Mg⁺², and Mn⁺² and is inhibited by the presence of monovalent salts such as NaCl and KCl. (Kunitz, J. Gen. Physiol. 1950:33:349-362; Campbell and Jackson, J. Biol. Chem. 1980; 255:3726-3735 DNase I also is strongly inhibited by globular actin (G-actin) (Lazarides and Lindberg, Proc. Natl. Acad. Sci. USA 1974:71:4742-4746).

DNase I is often used as a reagent for the removal of residual or unwanted DNA from solutions of RNA, e.g., during the purification of RNA from biological sources. In addition, DNase I is used in techniques for generating synthetic RNA, such as in in vitro transcription reactions, for generating cRNA, and in the enzymatic cleavage of double-stranded DNA in DNA footprinting assays to detect protein binding sites.

In such applications, it is desirable to have the ability to employ DNase I activity under the broadest possible conditions of use, for example, under conditions that may normally be inhibitory to the native enzyme. For example, recombinant DNase I enzymes have been engineered that retain significant activity in the presence of higher concentrations of NaCl, permitting the efficient digest of DNA in solutions of this salt. See, e.g., U.S. Patent Publication 20040219529; Pan and Lazarus, J. Biol. Chem. 1998; 273:11701-08. Other recombinant enzymes have been generated which are resistant to inhibition by G-action. See, e.g., Pan, et al. J Biol. Chem. 1998; 273(29):18374-81. An alternative approach to engineering novel enzymes is to compensate for decreased specific activity of DNase I by adding large quantities of a partially inhibited enzyme, thereby providing sufficient activity to digest DNA. See, e.g., as described in U.S. Pat. No. 6,218,531. This approach has certain practical disadvantages, including waste of a costly enzyme, an increased potential for carry-over of enzyme into downstream processes, as well as increased potential for adding contaminating RNase activity, which is frequently observed in DNase I preparations.

### SUMMARY OF THE INVENTION

In one embodiment, the invention relates to methods for use of DNase I under conditions which are normally inhibitory to the native enzyme and to compositions and kits for facilitating the methods.

In one embodiment, the invention relates to a composition comprising a DNase I-like enzyme and an organic solvent which is not glycerol, although the composition may additionally include glycerol. In one aspect, the organic solvent comprises an alcohol. In certain embodiments, the organic solvent is present in at least about 20% v/v in an aqueous solution comprising the DNase I-like enzyme, or at least about 40%, at least about 60% or up to about 99% v/v organic solvent. In certain aspects, the aqueous solution comprises a solution which would be inhibitory to the DNase I-like enzyme in the absence of organic solvent.

In one aspect, the DNase I-like enzyme comprises bovine pancreatic DNase I. In another aspect, the DNase I-like enzymes comprises a recombinant enzyme.

In certain aspects, the alcohol comprises a monohydroxyl alcohol, such as, for example, methanol, ethanol, isopropanol, butanol, isomers thereof, stereoisomers thereof, and combinations thereof.

In other aspects, the alcohol comprises a di-hydroxylic alcohol, such as, for example, ethane diol, propane diol, butane diol, isomers thereof, stereoisomers thereof, and combinations thereof.

In still other aspects, the alcohol comprises a tri-hydroxylic alcohol.

In certain aspects, the alcohol comprises a combination of one or more different monohydroxyl alcohols, di-hydroxylic or tri-hydroxlic alcohols. Although, in one aspect, the composition comprises at least one non-glycerol alcohol, the composition may additionally include glycerol.

In one embodiment, compositions such as discussed above are produced by dehydrating a DNase I-like enzyme (e.g., such as by lyophilizing the enzyme) and adding a solution comprising an organic solvent that is not glycerol.

In another embodiment, the invention relates to kits which comprise any of the compositions described above and an aqueous solution provided in a separate composition from the container, e.g., to modify the v/v concentration of organic solvent to aqueous solution in the composition.

In one aspect, the aqueous solution comprises a solution which is inhibitory to the DNase I-like enzyme in the absence of organic solvent.

In a further embodiment, the invention relates to a method comprising contacting a sample comprising a DNA molecule with a DNase I-like enzyme and a solution comprising an organic solvent that is not glycerol, although glycerol may be additionally present in the solution or in the sample. In certain aspects, the solution comprises a salt concentration inhibitory to the DNAse I-like enzyme in the absence of the organic solvent. In one aspect, the organic solvent comprises an alcohol, such as a monohydroxyl, di-hydroxylic, or trihydroxylic alcohol or combination thereof, as discussed above.

The sample can be a cell or tissue sample or a sample comprising at least partially purified nucleic acid molecules.

In one aspect, RNA is transcribed from the DNA molecules prior to digestion with the DNase I-like enzyme.

In another aspect, the sample comprises an at least partially double-stranded DNA molecule.

In a further aspect, the method comprises contacting the DNA molecule with the DNase I-like enzyme under conditions that digest 50% or greater of the DNA molecules to about 100 base pairs or less.

In still another aspect, the method comprises contacting the DNA molecule with the DNase I-like enzyme under the conditions to produce single-strand nicks in the DNA molecule. In a further aspect, the method comprises contacting the DNA molecule with the DNase I-like enzyme under conditions to produce double-strand nicks in the DNA molecule.

In another embodiment, the method comprises obtaining a nicked DNA molecule and contacting the molecule with at least one deoxyribonucleotide in the presence of a polymerase and/or ligase. In one aspect, the at least one deoxyribonucleotide is labeled.

In a further embodiment, the method further comprises contacting the DNA molecule with a DNA-binding protein prior to contacting with the DNase I-like enzyme. In one aspect, the DNA-binding protein is crosslinked to the DNA-binding protein. In another aspect, the method additionally comprises inactivating or removing the DNase I-like enzyme after contacting, and collecting DNA molecules bound to protein. Proteins can be removed from the DNA molecules, for example, by reversing crosslinking using heat, and the sequence of the DNA molecules can be characterized, for example, by binding the DNA molecules to an array.

In a further embodiment, the invention further provides a method of storing a DNase I-like enzyme comprising contacting a composition comprising a DNase I-like enzyme in the presence of an organic solvent solution that is not glycerol and storing the DNase I-like enzyme. In one aspect, the DNase I-like enzyme is lyophilized prior to contacting. In certain aspects, the DNase I-like enzyme is stored for at least 24 hours in the solution. In certain aspects, in addition to comprising an organic solvent that is not glycerol, the solution further comprises glycerol. In certain other aspects, the DNase I-like enzyme is stored at temperatures greater than 4° C., or at room temperature.

### BRIEF DESCRIPTION OF THE FIGURES

FIG. 1 is a graph illustrating the effects of isopropanol and NaCl on DNase I activity.

FIG. 2 is a bar graph illustrating the effects of different alcohols and salt on DNase I activity.

### DESCRIPTION OF THE INVENTION

Before describing the present invention in detail, it is to be understood that this invention is not limited to specific compositions, method steps, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” includes more than one biopolymer, and reference to “a voltage source” includes a plurality of voltage sources and the like.

Definitions

The following definitions are provided for specific terms that are used in the following written description.

The term “binding” refers to two molecules associating with each other to produce a stable composite structure under the conditions being evaluated (e.g., such as conditions suitable for RNA or DNA isolation). Such a stable composite structure may be referred to as a “binding complex”.

As used herein, the term “RNA” or “oligoribonucleotides” refers to a molecule having one or more ribonucleotides. The RNA can be single, double or multiple-stranded (e.g., comprise both single-stranded and double-stranded portions) and may comprise modified or unmodified nucleotides or non-nucleotides or various mixtures and combinations thereof.

As used herein, the term “DNA” or “deoxyribonucleotides” refers to a molecule comprising one or more deoxyribonucleotides. The DNA can be single, double or multiple-stranded (e.g., comprise both single-stranded, double-stranded, and triple-stranded portions) and may comprise modified or unmodified nucleotides or non-nucleotides or various mixtures and combinations thereof.

As used herein “complementary sequence” refers to a nucleic acid sequence that can form hydrogen bond(s) with another nucleic acid sequence by either traditional Watson-Crick or other non-traditional types (for example, Hoogsteen type) of base-paired interactions.

In certain embodiments, two complementary nucleic acids may be referred to as “specifically hybridizing” to one another. The terms “specifically hybridizing,” “hybridizing specifically to” and “specific hybridization” and “selectively hybridize to,” are used interchangeably and refer to the binding, duplexing, complexing or hybridizing of a nucleic acid molecule preferentially to a particular nucleotide sequence under stringent conditions. “Hybridizing” and “binding”, with respect to polynucleotides, are used interchangeably.

The term “reference” is used to refer to a known value or set of known values against which an observed value may be compared.

It will also be appreciated that throughout the present application, that words such as “cover”, “base” “front”, “back”, “top”, “upper”, and “lower” are used in a relative sense only.

As used herein, the term “solid phase” or “solid substrate” or “matrix” includes rigid and flexible solids. Examples of solid substrates include, but are not limited to, gels, fibers, whiskers, resins, microspheres, spheres, cubes, particles of other shapes, channels, microchannels, capillaries, walls of containers, membranes and filters.

As used herein, the term “silica-based” is used to describe SiO₂ compounds and related hydrated oxides and does not encompass silicon carbide compositions, which are described herein.

As used herein, a “nucleic acid binding material”, stably binds a nucleic acid (e.g., such as double-stranded, single-stranded, partially double-stranded, or triple-stranded DNA, RNA or modified form thereof). By “stably binds” it is meant that under defined binding conditions the equilibrium substantially favors binding over release of the subcellular component, and if the solid substrate containing a selected bound subcellular component is washed with buffer lacking the component under these defined binding conditions, substantially all the component remains bound. In particular embodiments the binding is reversible. As used herein, the term “reversible” means that under defined elution or release conditions the bound nucleic acid component of a sample is predominantly released from the nucleic acid binding material and can be recovered (e.g., in solution). In particular embodiments, at least about 10%, at least about 20%, at least about 50%, at least about 60%, at least 90%, or at least 95% of the bound nucleic acid component is released under the defined elution or release conditions.

As used herein, a “nucleic acid capture material” is one which preferably retains, traps, or remains associated with nucleic acids to remove a nucleic acid from solution. A nucleic acid capture material may, but does not necessarily bind to a nucleic acid molecule.

“Washing conditions” include conditions under which unbound or undesired components are removed from a module of a device described below.

The term “assessing” “inspecting” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

Additional terms relating to arrays and the hybridization of nucleic acids to such arrays may be found, for example, in U.S. Pat. No. 6,399,394 and U.S. Pat. No. 6,410,243.

The invention provides a method for expanding the range of use of a DNase I-like enzyme. As used herein, a “DNase I-like enzyme” is a natural, recombinant or synthetic enzyme, fragment thereof, or fusion protein thereof, that substantially nonspecifically cleaves single-stranded, double-stranded, triple-stranded, or partially single-stranded, double-stranded, or triple-stranded DNA molecules, or DNA:RNA hybrids to release mono-, di-, tri- and oligonucleotide products with 5′-phosphorylated and 3′-hydroxylated ends. As used herein, “substantially nonspecific cleavage” means that variability of cleavage at a given base usually does not vary substantially from that of bovine pancreatic DNase I.

In one aspect, a DNase I-like enzyme produces single-strand nicks (e.g., in the presence of Mg²⁺). In another aspect, a DNase I-like enzyme produces double-strand nicks (e.g., in the presence of Mn²⁺ and absence of Mg²⁺). In a further aspect, a DNase I-like enzyme comprises bovine pancreatic DNase I, EC:3.1.21.1 or an enzyme which comprises substantially the same specific activity of bovine pancreatic DNAse I. In one aspect, the DNase I-like enzyme is a recombinant enzyme. In certain aspects, a DNase I-like enzyme lacks an actin-binding domain but otherwise retains the salt sensitivity of native bovine pancreatic DNase I enzyme, e.g., loses about 50% or more activity in the presence of ≧100 mM of a monovalent salt such as NaCl or KCl.

In one embodiment, the invention provides a method of contacting a DNA molecule with a DNase I-like enzyme in the presence of a salt concentration inhibitory to the enzyme, e.g., a salt concentration in which the enzyme loses at least about 10%, at least about 20%, at least about 30%, at least about 40%, at least about 50%, or more of its activity. As used herein, the “activity” of a DNase I-like enzyme refers to a measure of ability of the DNase I-like enzyme to catalyze cleavage of a selected substrate over a selected period of time. While any of a number of assays can be used to monitor DNase I-like activity, in one aspect, an enzyme having a DNAse I-like activity has a specific activity of >10,000 units/mg, where one unit is defined as the amount of enzyme that increases the absorbance at A260 nm in a 1 cm path length at a rate of 0.001 units per min per ml of 0.05 mg/ml calf thymus DNA (Sigma) in the presence of 10 mM Tris-HCl, pH 8.0, 0.1 mM CaCl₂ and 1 mM MgCl₂ (see, e.g., Kunitz. J. Gen. Physiol. 1950; 33:349-362).

In one embodiment, the method comprises contacting the DNase I-like enzyme with a DNA template (e.g., single-stranded, double-stranded, partially double- or single-stranded DNA or a DNA:RNA hybrid) in the presence of an effective amount of organic solvent to permit digestion of at least about 50% of the DNA template in 15-30 minutes to oligonucleotides of 100 bases or less, 50 bases or less, 20 bases or less, 10 bases or less, or 3 bases or less. In one aspect, the organic solvent is not glycerol, although glycerol may be added as an additional organic solvent.

In one aspect, the organic solvent comprises an alcohol which is not glycerol, although glycerol may be provided as an additional alcohol. Exemplary alcohols include, but are not limited to low molecular weight alcohols, such as monohydroxyl alcohols, e.g., methanol, ethanol, isopropanol (e.g., 1- and 2-isopropanol) and butanol (e.g., 1- and 2-butanol). Other examples include, but are not limited to, di-hydroxylic alcohols, such as ethane diol, propane diol, butane diol, and the like. Still other examples include tri-hydroxylic alcohols.

In one aspect, an effective amount of an organic solvent comprises greater than approximately 20% v/v organic solvent, greater than about 45% v/v organic solvent, greater than about 50% v/v organic solvent, and up to about 99% v/v organic solvent. In one aspect, the DNase I-like enzyme retains its activity in the presence of at least about 10 mM of a monovalent salt such as NaCl or KCl, at least about 20 mM, at least about 30 mM, at least about 50 mM, at least about 100 mM, at least about 150 mM, or at least about 200 mM of the monovalent salt. At lower volumes of organic solvent, the lowest molecular weight alcohols may be preferred (e.g., such as methanol or ethanol).

The remainder of the solution in which DNase digestion takes place may comprise any standard buffer, e.g., comprising appropriate monovalent and/or divalent cations. In one aspect, a 1× DNase I digestion buffer comprises 10 mM Tris-HCl, pH 8.0, 10 mM MgSO₄, and 1 mM CaCl₂.

In certain aspects, the digestion buffer does not comprise a divalent cation such as Mg²⁺ or Ca²⁺.

DNA digestion can be performed in a variety of applications, e.g., to remove contaminating genomic DNA from an RNA sample, to degrade a DNA template in a transcription reaction, in a nick translation reaction or DNase I footprinting reaction. Methods for performing these techniques are known in the art.

The average size of the resulting DNA fragments generated by the method can be modulated by optimizing enzyme to substrate ratios and incubation time to suit a desired application.

In one embodiment, the invention further relates to a method comprising treating a sample of RNA to remove a substantial amount of gDNA while maintaining RNA integrity in a sample. In one aspect, the method comprises isolating RNA. In another aspect, the method comprises isolating RNA in a potentiating amount of organic solvent in an aqueous buffer (e.g., from 1% to 80% v/v aqueous buffer), e.g., an amount that results in optimal recovery of RNA compared to recovery in the absence of organic solvent and the presence of 100% aqueous buffer.

In one embodiment, a sample is homogenized in an extraction buffer. Sample sources include, but are not limited to, animals, plants, fungi (e.g., such as yeast), bacteria, and portions thereof. In one aspect, the animal can be a mammal, and in a further aspect, the mammal can be a human. Sample sources may additionally include virally infected cells, as well as transgenic animals and plants or otherwise genetically modified animals and plants. In addition, the sample can originate from experimental protocols, for example, from a polymerase chain reaction or from the products of an enzymatic reaction (e.g., a polymerization and/or transcription reaction).

In certain embodiments, samples are lysed before, during, or after homogenization. Suitable lysis solutions are known in the art. However, in one aspect, the lysis solution comprises a chaotropic salt, and/or additives to protect nucleic acids in the sample from degradation or reduced yield. Suitable salts include but are not limited to, urea, formaldehyde, ammonium isothiocyanate, guanidinium isothiocyanate, guanidinium hydrochloride, formamide, dimethylsulfoxide, ethylene glycol, tetrafluoroacetate, diamineimine, ketoaminimine, hydroxyamineimine, aminoguanidine hydrochloride, aminoguanidine hemisulfate, hydroxylaminoguanidine hydrochloride, sodium iodide, sodium perchlorate, and mixtures thereof. In another aspect, the lysis solution comprises one or more enzymes to facilitate disruption of cells in a sample. Suitable enzymes include, but are not limited to, a protease, lysozyme, zymolase, cellulase, and the like. In still other aspects, a lysis solution may include one or more agents for stabilizing nucleic acids, such as, but not limited to cationic compounds, detergents (e.g., SDS, Brij, Triton-X-100, Tween 20, DOC, and the like), chaotropic salts, ribonuclease inhibitors, chelating agents, DEPC, vanadyl compounds, and mixtures thereof. Examples of ribonuclease inhibitors can be found in Farrell R. E. (ed.) (RNA Methodologies: A Laboratory Guide for Isolation and Characterization, Academic Press, 1993) and Jones, P. et al. (In: RNA Isolation and Analysis, Bios Scientific Publishers, Oxford, 1994). In one aspect, RNAlater® (Ambion Inc., Austin, Tex., U.S. Pat. No. 6,204,375) is used as an RNAse inhibitor. In one embodiment, a lysis solution comprising at least about 4M guanidine isothiocyanate (e.g., from about 4M to about 6M) guanidine isothiocyanate is used in a Tris buffer of from about pH 6-8 (e.g., about pH 6.6 to about 7.5), EDTA (e.g., about 10 mM) and optionally, about 0.5-1% β-mercaptoethanol is used.

In still another aspect, the lysis solution comprises an amount of salt, which is typically inhibitory to the activity of a DNase I-like enzyme, e.g., at least about 10 mM of a monovalent salt such as NaCl or KCl, at least about 20 mM, at least about 30 mM, at least about 50 mM, at least about 100 mM, at least about 150 mM, or at least about 200 mM of the monovalent salt.

Mechanical homogenization can be performed using methods known in the art, e.g., such as by using a rotor-stator homogenizer, such as by grinding in a mortar and pestle with liquid nitrogen, mechanical disruption with a tissue homogenizer, such as a Polytron® or Omniprobe® homogenizer, manual homogenization (e.g., with a Dounce homogenizer), shaking the sample in a container with metal balls, or vortexing vigorously. Additionally, or alternatively, samples can be homogenized by ultrasonic disruption. In one aspect, homogenization is done in a high chaotrope concentration solution effectively lysing cells and destroying cellular enzymatic activity, such as the activity of nucleases, until a desired nuclease can be added under controlled conditions.

In one aspect, a homogenized sample is transferred to a device according to the invention for contacting with a separation module which preferentially retains genomic DNA and cellular debris while allowing RNA molecules to pass through.

As used herein, the term “module” refers to an element or unit in the device that may or may not be removable from the device. In one aspect, the device comprises a housing having an open end and comprises walls defining a lumen into which the module fits. In another aspect, the device comprises a closed bottom end. The separation module may be removable from the housing or an integral part of the housing or some combination thereof. The shape and dimensions of the housing may vary. However, in one embodiment, the housing is shaped like a tube or column. In another aspect, the housing is shaped like a tube and the separation module is provided in the form of a column that fits into the tube, the remaining space defining a collection compartment or chamber for receiving flow through from the separation module.

In certain aspects, a plurality of device housings is provided in a holder or container or rack and a plurality of separation modules (e.g., columns) may be inserted into the lumen of each of the housings. In one aspect, the plurality of device housings is provided as a single unit (e.g., molded as a single unit from a plastic or other suitable material) comprising a plurality of lumens for receiving a plurality of columns.

Individual separation modules may be separated from each other one at a time, e.g., by unscrewing or snapping apart. Likewise, the housing may be made from a variety of materials, including but not limiting to, a polymeric material such as plastic, polycarbonate, polyethylene, PTFE, polypropylene, polystyrene and the like.

In one embodiment, the separation module separates two different types of biopolymers from each other. In one aspect, the separation module separates DNA (such as genomic DNA) from RNA (e.g., such as total cellular RNA). In another aspect, the separation module comprises one or more filters or layers of beads or other type of matrix. For example, in one aspect, the separation module comprises a porous material. Suitable materials for fabricating the module include, but are not limited to, glass fibers or borosilicate fibers, silica gels (which may be further treated using chaotropic salts), polymers (e.g., beads, filters, membranes, fibers) and the like.

In one aspect, the separation module comprises a fiber material that demonstrates particle retention in the range of about 0.1 μm to about 10 μm diameter equivalent. The fibers can have a thickness ranging from about 50 μm to about 2,000 μm. For example, in one aspect, a fiber filter has a thickness of about 500 μm. The specific weight of a fiber filter can range from about 75 g/m² up to about 300 g/m2. Multiple fiber layers are envisaged to be within the scope of this invention. The fiber may, optionally, comprise a binder, e.g., for improving handling of the fiber or for modifying characteristics of a composite fiber (i.e., one which is not pure borosilicate). Examples of binders include, but are not limited to, polymers such as acrylic, acrylic-like, or plastic-like substances. Although it can vary, typically binders may represent about 5% by weight of the fiber filter.

The pore size of the filter may be uniform or non-uniform. Where a plurality of filters are used, the pore size of each filter may be the same or different. In another aspect, suitable pore sizes may range from about 0.1 μm to about 2 mm.

In a particular aspect of this invention, the separation module comprises at least one layer of fiber filter material along with a retainer ring that is disposed adjacent to a first surface of the fiber filter material that securely retains the layer(s) of fiber filter material so that they do not excessively swell when sample is added. In one aspect, a frit is provided which is disposed adjacent to a second surface of the fiber filter material. The frit may assist in providing support so that the materials of the filter fibers do not deform. In one aspect, the frit is composed of polyethylene of about 90 μm thick. In certain aspects, the separation module comprises at least two layers of filter material, at least three layers, at least four layers, at least five layers, at least six layers, at least seven layers, at least 8 layers, at least 9 layers, or at least 10 layers.

In one embodiment, the separation module comprises Whatman GF/F Glass Fiber Filters (cat no. 1825-915) (available from Fisher Scientific, Atlanta, Ga.) or an eq equivalent material. Multiple layers (of the large sheets or disks supplied) may be punched, for example, with a 9/32″ hand punch (McMaster-Carr, Chicago, Ill.) and placed into a spin column (Orochem, Westmont, Ill.) fitted with a 90 μm polyethylene frit (Porex Corp., Fairburn, Ga.) on which the fibers may rest. The filter materials may be secured in the column with a retainer ring on top of the filter materials to prevent excessive swelling of the fibers or movement during centrifugation. In one aspect, the separation module that is used is the prefiltration column available in Agilent's Total RNA Isolation Mini Kit prefiltration column (Catalog #5185-6000) from Agilent Technologies, Inc. (Palo Alto, Calif.).

In one aspect, the separation module does not comprise a matrix for anion exchange.

Flow-through from the column comprising RNA molecules, obtained after centrifugation or application of pressure to the device, is collected within the collection module of the device. In one aspect, a sample is applied to the separation module in a solution comprising a chaotropic agent and an organic solvent, such as an alcohol, in the range of about 40-60% by volume. As discussed above, exemplary alcohols include, but are not limited to, monohydroxyl alcohols, e.g., methanol, ethanol, isopropanol (e.g., 1- and 2-isopropanol) and butanol (e.g., 1- and 2-butanol). Other examples include, but are not limited to, di-hydroxylic alcohols, such as ethane diol, propane diol, butane diol, and the like. In another aspect, a DNAse I-like enzyme is added to the solution in suitable quantities to convert greater than about 50%, greater than about 60%, greater than about 70%, greater than about 80%, greater than about 90%, or greater than about 95% of genomic DNA in the sample to fragments of 20 bp or less, e.g., 0.2-200 units per μg of DNA.

In a certain aspects, sample is applied to the separation module and the separation module is washed with a solution comprising an organic solvent in the range of about 50-100% v/v. However, in another aspect, the organic solvent is provided in a potentiating amount to provide for optimal recovery of RNA from a sample being treated with the DNase I-like enzyme. In still other aspects, the aqueous component of the wash solution comprises a concentration of a salt which is typically inhibitory to a DNAse I-like enzyme, e.g., at least about 10 mM of a monovalent salt such as NaCl or KCl, at least about 20 mM, at least about 30 mM, at least about 50 mM, at least about 100 mM, at least about 150 mM, or at least about 500 mM of the monovalent salt. In a further aspect, a DNase I-like enzyme is added to the solution in a suitable quantity as described above.

In alternative, or additional aspects, the solid phase material within the separation module is impregnated with a DNAse I-like enzyme (e.g., in a lyophilized or dried form) and can be activated by contacting a sample to the solid phase material in a solution comprising at least about 40% organic solvent, as described above. In certain aspects, the solution additionally comprises a chaotropic salt.

RNA in the flow-through from the separation module can be collected within the lumen of the housing between the module and the closed end of the same or a different device (i.e., the separation module can be transferred to the housing of a different device). This portion of the device forms the “collection module.” RNA collected in the collection module can be removed from the collection module for further processing steps. Additionally, or alternatively, processing steps may occur in the collection module. While there may generally be sufficient organic solvent and salt in the wash solution or added to the lysis solution to precipitate RNA as it is passing through the separation module, additional organic solvent may be added in the collection module, e.g., to wash a pelleted RNA sample or further enhance the precipitation process.

In one aspect, the separation module is provided in the form of a column that fits into the lumen defined by the walls of the device housing and the collection module is formed in the space between the column and the closed bottom end of the housing. Removing the column from the device provides access to the collection module. Alternatively, the collection module may be removed from the device (e.g., by snapping off or twisting). In one aspect, the closed bottom end may comprise a cap or cover which may be removed to obtain collected RNA-enriched material.

In still other embodiments, a flow through from a separation module is collected in a collection module and transferred to a new collection module which comprises molecules (e.g., in the form of a membrane, matrix, gel, particles, beads, filter, and the like) for specifically binding RNA or for capturing or trapping RNA (e.g., such as precipitated RNA), for example, to remove any remaining contaminants in the solution or to further concentrate a sample. For example, an RNA capture membrane may be provided as part of the collection module to facilitate the collection of the RNA precipitate, washing of the collected precipitate (reducing wash volumes and centrifugation times) and re-suspension and elution or release of RNA. Alternatively, the flow through may be collected directly in a collection module, which comprises the RNA-binding molecules or other RNA-capture material (e.g., such as a matrix for trapping precipitated RNA).

In certain aspects, the collection module includes material that reversibly captures RNA. Suitable nucleic acid capture materials are known in the art and include, but are not limited to, SiO₂-based materials or silicon carbide (see, e.g., U.S. Pat. Nos. 6,177,278 and 6,291,248). As an alternative to silicon carbide, silica materials such as glass particles, glass powder, silica particles, glass microfibers, diatomaceous earth, and mixtures of these compounds may be employed. Nucleic acid capture materials may be combined with chaotropic salts to isolate nucleic acids. In one aspect, a nucleic acid capture material comprises a silicon carbide matrix, e.g., such as silicon carbide fibers or whiskers. In another aspect, the capture materials comprise silica carbide whiskers which comprise a comparatively high specific surface area material, greater than about 0.4 m²/g, greater than 1 m²/g, greater than 2 m²/g, greater than 3 m²/g or about 3.9 m²/g as measured by surface Nitrogen absorption.

In another aspect, the collection module comprises one or more polymeric membranes, examples of which include, but are not limited to, polysulfone, e.g., such as a BTS membrane (Pall Life Sciences), PVDF, nylon, nitrocellulose, PVP (poly(vinyl-pyrrolidone)), MMM filters (Pall Life Sciences, available from VWR, Pittsburg, Pa.) and composites thereof. In one aspect, the membrane is a composite of Polysulfone and PVP. In another aspect, the binding material comprises an asymmetric membrane with pores that gradually decrease in size from the upstream side to the downstream side. In one aspect, the membrane comprises pore sizes from about 0.1 μm to 100 μm. In another aspect, the membrane comprises pore sizes of from about 0.1 μm to 5 μm, or from about 0.1 μm to about 10 μm, or from about 0.4 μm to about 0.8 μm. In still another aspect, the binding material comprises a hydrophobic and/or hydrophilic material. Glass fiber filters, such as used in the separation module can also be used.

In one embodiment of the present invention, the collection module comprises an isolation column comprising an inlet and an outlet between which lies a chamber comprising a single or multiple layers of a polymeric membrane, examples of which include polysulfone, PVP (Poly(vinylpyrrolidone)), MMM membrane (Pall Life Science), BTS, PVDF, nylon, nitrocellulose, and composites thereof. A retainer ring and a frit can be disposed about the membrane(s) to retain them within the collection module. For example, a retainer ring may be disposed proximal to the inlet while a frit may be disposed proximal to the outlet.

In one aspect, the column comprises an asymmetric porous membrane comprising of polysulfone and polyvinylpyrrolidone. In one aspect, the membrane comprises a first surface and a second surface, the first surface having pores which are larger than the pores on the second surface. For example, in one aspect, the first surface has 30-40 μm diameter pores and the second surface has 0.1-0.10 μm diameter pores, or 0.4-0.8 μm diameter pores. In another aspect, the membrane comprises intermediate sized pores between the first and second surface. In still another aspect, the larger diameter pores are on the upper side of the membrane while the smaller diameter pores (proximal to the collection module of the device) are on the lower surface.

In one aspect, the matrix or membrane is substantially insoluble at elevated pHs and reversibly absorbs nucleic acids. In another aspect, the matrix is an MMM membrane or plurality of MMM membranes.

Examples of RNA capture materials additionally include, but are not limited to, various types of silica, including glass and diatomaceous earth. In some aspect, binding materials include binding moieties stably associated with a solid phase, such that RNA molecules will bind to the solid phase by virtue of this association. RNA-capture materials include cation exchange groups such as carboxylates, and hydrophobic interaction groups. Thus, examples of solid phase nucleic acid capture materials also include silica particles, magnetic beads coated with silica, and resins coated with cation exchange groups, hydrophobic interaction groups, dyes, and the like. However, in a further aspect, the RNA capture material does not comprise silica.

In certain aspects, the RNA capture material comprises a porous or semi-porous of fibrous material which captures precipitated RNA within its pores/between its fibers. It should be noted that an RNA capture material also may comprise an RNA-binding material and that the mechanism by which RNA is selectively retained within the capture material is not a limiting feature of the invention.

Although in one aspect, the separation module substantially removes all of genomic DNA in a sample, in certain aspects, DNase I-like enzymes are additionally added to the collection module, e.g., in solution or in impregnated in an RNA-capture material such as described above. In certain aspects, digestion by a DNAse I-like enzyme within the collection module occurs in the presence of an at least about 40% v/v solution of organic solvent as described above.

In still other aspects, however, a cell or tissue lysate is contacted to the separation module in a less than 20% solution of organic solvent, such that RNA is not precipitated as it passes through the separation module. RNA can be precipitated and additionally treated with a DNAse I-like enzyme in an at least about 20% solution of organic solvent within the collection module using RNA-capture materials as described above. In still other aspects, it is desirable not to add a DNase-I like enzyme to the separation module, e.g., where the separation module is later used to collect genomic DNA, for example, in methods for obtaining both RNA and genomic DNA in a sample.

RNA eluted or released from RNA-capture materials in the collection module can be precipitated (e.g., in an amount of solvent which further comprises a DNAse I-like enzyme) and pelleted by centrifugation (e.g., a spin step of 30 seconds at room temperature at 16,000 g). Pelleted nucleic acids may be resuspended, for example, after washing at least once, or at least twice, with a wash solution, for example, such as 25 mM Tris-HCl pH 7.5, 80% ethanol. After a final wash, pelleted nucleic acids are resuspended in a suitable buffer, for example, H₂O or TE.

The quality and/or quantity of nucleic acids collected may be evaluated and optimized using methods well known in the art, such as obtaining an A260/A280 ratio, evaluating an electrophoresed sample, or by using Agilent Technologies® RNA 6000 Nano assay (part no. 5065-4476) on the Agilent Technologies® Bioanalyzer 2100 (part no. G2938B, Agilent Technologies, Inc., Palo Alto, Calif.) as per manufacturer's instructions.

As discussed above, in addition to RNA isolation, organic solvent/aqueous solutions according to the invention can be used with DNase I-like enzymes in a variety of applications.

In one embodiment, a method according to the invention comprises providing a DNA template encoding an RNA product and contacting the DNA template with an RNA polymerase in the presence of suitable amounts of ribonucleotides under conditions for performing an in vitro transcription reaction. The remaining DNA template is removed by contacting the solution with an amount of organic solvent to produce a solution that is suitable for maintaining the activity of a DNAse I-like enzyme despite the presence of an amount of salt that is typically inhibitory to that DNase I-like enzyme. In one aspect, the solution after contacting with organic solvent comprises at least about 20% v/v organic solvent and the DNA template is incubated in the solution for a suitable amount of time (e.g., 10-15 minutes at 25° C. to about 37° C., or higher, e.g., if using a thermostable DNase I-like enzyme). RNA transcripts may be collected by centrifugation, optionally, after adding additional amounts of organic solvent. In one aspect, RNA transcripts are contacted to an RNA-binding matrix, such as described above.

In another embodiment, an organic solvent/aqueous solution according to the invention is used in a nick-translation reaction to label a DNA molecule. In one aspect, the method comprises providing a DNA template and a DNase I-like enzyme in the presence of at least about 40% of an organic solvent (v/v) as described above and incubating the enzyme under conditions for introducing nicks into the DNA template. In another aspect, the aqueous component of the solution comprises an amount of salt that is typically inhibitory of the DNase I-like enzyme. Nicked DNA is then precipitated and contacted with deoxyribonucleotides, a DNA polymerase such as E. coli DNA polymerase I, and ligase (e.g., such as T4 ligase), resuspended in buffer and incubated under conditions suitable for DNA polymerization of the nicked template. In certain aspects, the DNAse-I like enzyme is inactivated prior to precipitation, e.g., by the addition of additional solvent, by the addition of EDTA and/or by heating the enzyme (e.g., at 70° C. for about 5 minutes). Nick-translated and ligated DNA can be separated from unincorporated dNTPs using methods known in the art, e.g., by chromatography through a column of Sephadex G-50 or by spun-column chromatography.

In a further embodiment, an organic solvent/aqueous solution according to the invention is used in location analysis. In one aspect, proteins that bind genomic DNA (e.g., such as proteins in a cell) are crosslinked to the DNA, e.g., by formaldehyde or another suitable fixative or condition. In certain aspects, the proteins are predefined, e.g., one or more known proteins are added in vitro to a solution of DNA. In other aspects, the proteins are from a complex sample, such as a cellular lysate. The resulting mixture, which includes DNA bound by protein and DNA which is not bound by protein is exposed to a DNase I-like enzyme in an organic solvent/aqueous solution at a final concentration which is at least about 20% v/v organic solvent for a sufficient amount of time to generate DNA fragments, including some which are bound by protein. Unbound DNA, digested to sizes of about 20 bases or less by the DNAse I-like enzyme can be removed, e.g., via a spin column.

Protein-DNA complexes can be contacted with protein-binding molecules, optionally, after pelleting by centrifugation and resuspending the complexes in an appropriate buffer for sorting particular protein-DNA complexes. Alternatively, complexes can be sorted directly in organic solvent-containing buffer.

Suitable sorting methods include, but are not limited to, immunoprecipitation or affinity-based methods which comprise the use of predefined protein-binding molecules (e.g., antibodies, affibodies, aptamers and the like) stably associated with a solid support. Crosslinked proteins may subsequently be removed from DNA, e.g., by heating at a temperature that also inactivates the DNase I-like enzyme, and the remaining fragments can be detected by a suitable method to identify the genomic region to which the proteins bind. For example, fragments can be sequenced or applied to an array for binding to a nucleic acid probe which can be used to identify and characterize the fragment as described in U.S. Pat. No. 6,410,243. In certain aspects, fragments are amplified prior to application to an array, e.g., by a substantially unbiased amplification method such as multiple-strand displacement amplification or through the use of primer binding sites ligated to the ends of the fragments as described in U.S. Pat. No. 6,410,243.

Generally, a DNase I-like enzyme can be contacted to a DNA template in an organic solvent/aqueous solution according to the invention for use in any application in which a DNase I-like enzyme is used. The applications described above are not limiting and others will be obvious to those of skill in the art based on the disclosure herein and are encompassed within the scope of the invention.

In an additional embodiment, the invention further relates to storage-stable solutions of a DNase I-like molecule comprising a DNase I-like enzyme in an about 20% or greater v/v solution of an organic solvent (including up to about 100%) which is not glycerol, though glycerol may be added as an additional component of the solution. Before use, an aqueous solution comprising sufficient water or buffer to produce an at least about 20% to 99% v/v solution of organic solvent may be added along with a suitable template for digestion of the template as described above. In certain aspects, a sufficient amount of water to provide a potentiating amount of organic solvent for isolating RNA from a sample is provided. In one aspect, the DNase I-like enzyme is lyophilized or otherwise dehydrated prior to contacting with the organic solvent.

In further embodiments, the invention relates to kits comprising a DNase I-like molecule, an organic solvent and, optionally, an aqueous solution. In one aspect, the organic solvent comprises an alcohol, which can include, but is not limited to, a monohydroxyl alcohol, e.g., methanol, ethanol, isopropanol (e.g., 1- and 2-isopropanol) and butanol (e.g., 1- and 2-butanol), a di-hydroxylic alcohol, such as ethane diol, propane diol, butane diol, and the like, or a combination thereof. In one aspect, the organic solvent and aqueous solution are mixed to provide a final volume which is at least about 20% to about 99% of organic solvent. In another aspect, the organic solvent is present at a higher concentration, e.g., up to about 100% v/v, and can be diluted by an aqueous solution, which is optionally included in the kit. In certain aspects, the DNase I-like enzyme is provided in an organic solvent, e.g., in a storage-stable form, as described above, or is provided in a ready-to-use form, e.g., in the presence of an amount of aqueous solution that permits DNA digestion. In certain aspects, the aqueous solution comprises an amount of salt that is typically inhibitory to the DNase I-like enzyme. In still other aspect, the kit comprises a device comprising a separation and/or collection module as described above. In certain aspects, the separation and/or collection module comprise a solid phase, which is impregnated with a DNase I-like enzyme and, optionally, an organic solvent.

### EXAMPLE

The invention is demonstrated further by the following illustrative examples which illustrate the invention but is not intended to limit its scope.

DNase I-Like Activity Determinations.

The enzymatic hydrolysis of calf thymus genomic DNA was assayed using a method modified from that described by Desai and Shankar (Eur. J. Biochem., 2000; 267: 5123-5135). The standard reaction mixture was 0.1 mL volume, containing 30 μg of sonicated native calf thymus genomic DNA (Sigma, St. Louis Mo., #D-3664), in a buffer solution composed of 100 mM Tris HCl(pH 8.0)/10 mM MgSO4/1 mM CaCl₂, with appropriately diluted DNase I (usually 0.02-1.0 Enzyme Units, as defined below). The reaction was initiated by the addition of enzyme, with incubation at room temperature for a defined period of time (usually 10-20 minutes), after which the reaction was terminated by rapid sequential addition of 0.1 mL of 1 mg/mL Bovine Serum Albumin (Sigma, #A3803) and 1.0 mL of ice cold 2% (v/v) Perchloric Acid. The terminated reaction mixture was vortex mixed, then chilled on ice for 20-30 minutes, follwed by centrifugation at 16,000×g for 10 minutes at 4 degrees centrigrade. The clarified supernatant contains acid-soluble oligonucleotides liberated by the action of DNase I-like activity, at concentrations determined using absorbance measurements determined in a 1 cm pathlength cell in the Agilent 8453 spectrophotometer (Agilent Technologies, Wilmington Del.). A molar extinction coefficient of 10,000 M⁻¹cm⁻¹ was employed for oligonucleotide concentration estimation in the acidic solution. Unit activity under these assay conditions is defined as umol of acid-soluble oligonucleotides generated per minute at 25 degrees centigrade. In the experiments shown in the examples that follow, bovine pancreatic DNase I is employed to illustrate the effects of various manipulations on the activity of this enzyme. Typically, the reaction employs approximately 0.1 enzyme units, as defined by the Kunitz assay, as discussed above (see Kunitz. J. Gen. Physiol. 1950; 33:349-362).

### Example 1

### DNase Activity in the Presence of Isopropanol and Salt

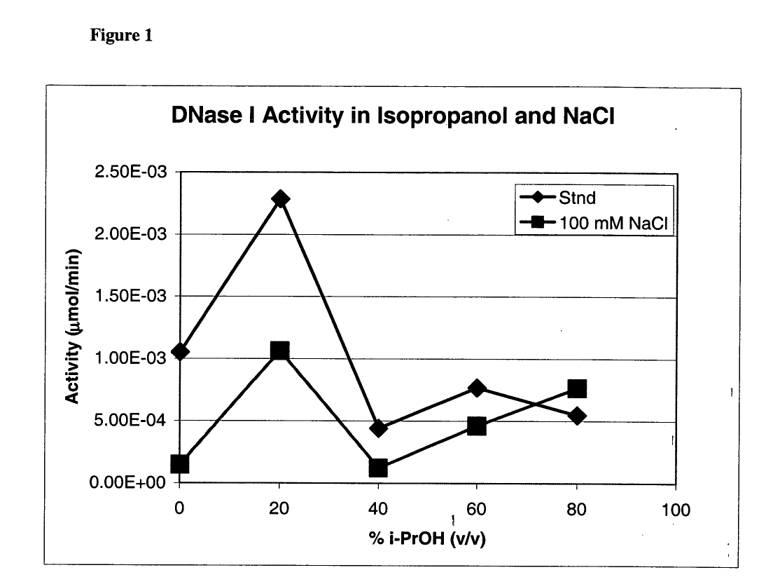

Bovine pancreatic DNase I (Fermentas, #EN0523) was assayed for gDNA hydrolytic activity, as described above, using the Standard reaction conditions, but modified by the inclusion of various quantities of iso-propanol (i-propanol, 2-propanol), with or without the further inclusion of sodium chloride at 100 mM. As shown in FIG. 1, the activity of bovine pancreatic DNase I activity is potentiated in the presence of 20% i-propanol, more than doubling the activity of the enzyme relative to standard contions. At higher concentrations of this alcohol, the activity decreases to about 50% of the standard condition, but in no case is eliminated, at concentrations up to, and above, 80% i-propanol. Again referring to FIG. 1, DNase activity is significantly inhibited by the presence of sodium chloride, with about 14% relative activity remaining on inclusion of 100 mM of this salt. The addition of 20% i-propanol to the salt-containing solution relieves the salt inhibition, bringing the activity of the enzyme to 101% of the Standard activity level. The addition of i-propanol at higher levels is observed to exhibit a biphasic pattern, with decreasing activity at 40%, thereafter increasing the DNase I activity. In no case was the enzyme completely inhibitied, and at high salt and high alcohol content, greater than 50% of the enzyme activity is observed.

### Example 2

### DNase Activity in the Presence of Different Alcohols and Salt

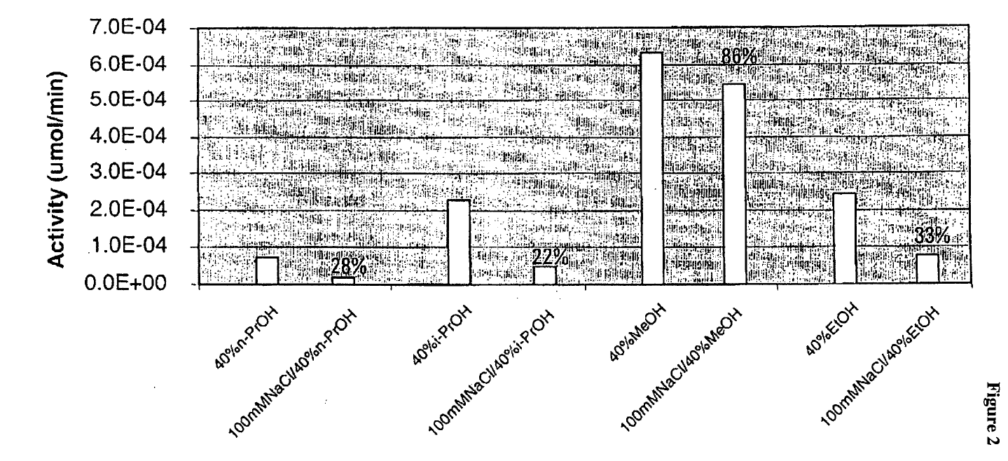

FIG. 2 presents the relative activities of pancreatic DNase I in the presence of various organic alcohol solvents, present at 40% (v/v) in the Standard reaction, with and without addition of 100 mM sodium chloride. It is clear that at this fixed concentration of alcohol, there are specific and significant differences in the inhibitory effects of the alcohols. The least inhibitory alcohol, at this concentration, is methanol, which shows about 72% activity of the Standard conditions (8.9×10⁻⁴ enzyme units). By comparison, n-propanol (normal-propanol, 1-propanol) at 40% concentration in the reaction retains about 10% of activity observed under Standard assay conditions. Ethanol and and i-propanol at 40% are observed to be about equally inhibitory, retaining 40% of the activity seen in Standard reaction conditions. Referring to the data of FIG. 2, all of the alcohol-containing reactions exhibited inhibition of the activity of DNase I on the addition of 100 mM sodium chloride to the reaction medium, but the degree of inhibition was observed to be significantly dependent on the alcohol present. At this concentration of alcohol, methanol supported the best retention of DNase activity on addition of salt, with 86% of the activity seen in 40% methanol alone, and about 62% of the activity seen in Standard reaction conditions. The presence of both n-propanol and 100 mM sodium chloride yielded the least enzyme activity, whereas i-propanol least prevented the inhibition of the enzyme by salt. The results for ethanol were intermediate, with about a three-fold reduction of enzyme activity induced by the addition of salt to the reaction condition that included 40% ethanol. Nevertheless, all of the alcohol/sodium chloride reaction mixtures supported some degree of DNase activity, although certain mixtures were poor, for example, n-propanol/sodium chloride supported less than 5% of the standard reaction condition activity of DNase I.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising a DNase I-like enzyme and an organic solvent which is not glycerol.

2. The composition of claim 1, wherein the organic solvent comprises an alcohol.

3. The composition of claim 1, wherein the organic solvent is present in at least about 20% v/v of a solution comprising the DNase I-like enzyme.

4. The composition of claim 1, wherein the organic solvent is present in at least about 60% v/v of a solution comprising the DNase I-like enzyme.

5. The composition of claim 1, wherein the DNase I-like enzyme comprises bovine pancreatic DNase I.

6. The composition of claim 1, wherein the DNase I-like enzymes comprises a recombinant enzyme.

7. The composition of claim 2, wherein the alcohol comprises a monohydroxyl alcohol.

8. The composition of claim 7, wherein the alcohol is selected from the group consisting of methanol, ethanol, isopropanol, butanol, isomers thereof, stereoisomers thereof, and combinations thereof.

9. The composition of claim 2, wherein the alcohol comprises a di-hydroxylic alcohol.

10. The composition of claim 9, wherein the alcohol is selected from the group consisting of ethane diol, propane diol, butane diol, isomers thereof, stereoisomers thereof, and combinations thereof.

11. The composition of claim 1 comprising at least about 99% organic solvent.

12. The composition of claim 1, produced by dehydrating a DNase I-like enzyme and adding a solution comprising an organic solvent that is not glycerol.

13. The composition of claim 12, wherein the DNase I-like enzyme is lyophilized.

14. A kit comprising the composition of claim 11 and an aqueous solution provided in a separate container from the composition.

15. The kit of claim 14, wherein the aqueous solution comprises a solution which is inhibitory to the DNase I-like enzyme in the absence of organic solvent.

16. A kit comprising a DNase I-like enzyme and an organic solvent which is not glycerol in separate containers.

17. The kit of claim 16, wherein the kit further comprises an aqueous solution which is optionally, in a separate container from the organic solvent.

18. The kit of claim 17, wherein the aqueous solution comprises a solution which is inhibitory to the DNase I-like enzyme in the absence of organic solvent.

19. The composition of claim 1, further comprising an aqueous solution which would be inhibitory to the DNase I-like enzyme in the absence of organic solvent.

20. The composition of claim 19, wherein the aqueous solution comprises at least about 10 mM of a monovalent salt.

21. The kit of claim 15, wherein the aqueous solution comprises at least about 10 mM of a monovalent salt.

22. The kit of claim 18, wherein the aqueous solution comprises at least about 10 mM of a monovalent salt.

23. A method, comprising:
contacting a sample comprising a DNA molecule with a DNase I-like enzyme and a solution comprising an organic solvent that is not glycerol.

24. The method of claim 23, wherein the solution comprises a salt concentration inhibitory to the DNAse I-like enzyme in the absence of the organic solvent.

25. The method of claim 24, wherein the organic solvent comprises an alcohol.

26. The method of claim 25, wherein the alcohol comprises a monohydroxyl alcohol.

27. The method of claim 26, wherein the alcohol is selected from the group consisting of methanol, ethanol, isopropanol, butanol, isomers thereof, stereoisomers thereof, and combinations thereof.

28. The method of claim 27, wherein the alcohol comprises a di-hydroxylic alcohol.

29. The method of claim 28, wherein the alcohol is selected from the group consisting of ethane diol, propane diol, butane diol, isomers thereof, stereoisomers thereof, and combinations thereof.

30. The method of claim 23, wherein the sample is a cell or tissue sample.

31. The method of claim 23, wherein RNA is transcribed from the DNA molecules prior to digestion with the DNase I-like enzyme.

32. The method of claim 23, wherein the sample comprises an at least partially double-stranded DNA molecule.

33. The method of claim 23, comprising contacting the DNA molecule with the DNase I-like enzyme under conditions that digest 50% or greater of the DNA molecules to about 100 base pairs or less.

34. The method of claim 23, comprising contacting the DNA molecule with the DNase I-like enzyme under the conditions to produce single-strand nicks in the DNA molecule.

35. The method of claim 23, comprising contacting the DNA molecule with the DNase I-like enzyme under conditions to produce double-strand nicks in the DNA molecule.

36. The method of claim 34, further comprising obtaining a nicked DNA molecule and contacting the molecule with at least one deoxyribonucleotide in the presence of a polymerase and/or ligase.

37. The method of claim 36, wherein the at least one deoxyribonucleotide is labeled.

38. The method of claim 23, wherein the method further comprises contacting the DNA molecule with a DNA-binding protein prior to contacting with the DNase I-like enzyme.

39. The method of claim 38, wherein the DNA-binding protein is crosslinked to the DNA-binding protein.

40. The method of claim of claim 38, further comprising inactivating or removing the DNase I-like enzyme after contacting, and collecting DNA molecules bound to protein.

41. The method of claim 40, comprising removing protein from the DNA molecules.

42. The method of claim 41, further comprising characterizing the sequence of the DNA molecules.

43. The method of claim 42, comprising contacting the DNA molecules to an array.

44. A method of storing a DNase I-like enzyme comprising contacting a composition comprising a DNase I-like enzyme in the presence of an solution comprising an organic solvent that is not glycerol and storing the DNase I-like enzyme.

45. The method of claim 44, wherein the composition comprising the DNase I-like enzyme is lyophilized prior to contacting.

46. The method of claim 44, wherein the DNase I-like enzyme is stored for at least 24 hours in the solution.

47. The method of claim 44, wherein the DNase I-like enzyme is stored at room temperature.

48. The composition of claim 2, wherein the organic solvent comprises a tri-hydroxylic alcohol.

49. The method of claim 23, wherein the organic solvent comprises a tri-hydroxylic alcohol.

50. The composition of claim 1, wherein the composition additionally comprises glycerol.

51. The method of claim 23, wherein the solution additionally comprises glycerol.

52. The method 44, wherein the solution additionally comprises glycerol.
